(19) United States
(12) Patent Application Publication
(10) Pub. No.: US 2019/0374478 A1
(43) Pub. Date: Dec. 12, 2019

(54) Ultrastable Gold Nanoparticles For Drug Delivery Applications And Synthesis Thereof

(71) Applicant: Université Laval, Québec (CA)

(72) Inventors: Élodie Boisselier, Québec (CA); Mathieu Ouellette, Québec (CA); Vincent Pernet, Québec (CA); Mahmoud Omar, Québec (CA)

(21) Appl. No.: 16/464,643

(22) PCT Filed: Dec. 6, 2017
(86) PCT No.: PCT/CA2017/051474

§ 371 Date: May 28, 2019

Related U.S. Application Data
(60) Provisional application No. 62/430,592, filed on Dec. 6, 2016.

(51) Int. Cl. A61K 9/51; A61K 9/00; A61K 31/5575; A61K 31/5377; A61P 27/06

(52) U.S. Cl. CPC A61K 9/5115; A61K 9/0048; A61P 27/06; A61K 31/5575; A61K 31/5377; A61K 9/5146

(57) ABSTRACT

Herein presented are gold nanoparticles (AuNPs) used for the transport of medications to mucous membranes. The AuNPs are ultra-stable in that they withstand freeze-drying and heating treatments without noticeable change in their structure. In addition, they interact with mucous membranes, and therefore allow transport of certain drugs to these mucous membranes, allowing higher exposure time of the drugs, hence decreased dosing schedule of administration.

### RELATED APPLICATION

This application claims priority to U.S. patent application Ser. No. 62/430.592 filed on Dec. 6, 2016, the content of which is incorporated herein by reference in its entirety for all purposes.

### FIELD OF THE INVENTION

The present invention relates to ultrastable gold nanoparticles (AuNPs) used for the transport of medication to mucous membranes. The interaction between the mucous membrane and the AuNPs allows greater exposure time of the drug, hence allowing decreased dosing schedule of administration. The present invention also relates to a method of synthesis of such AuNPs.

### BACKGROUND OF THE INVENTION

Gold aggregate is one of the most studied particles due to its unique properties that allow its use in diverse areas. Gold nano-aggregates can be divided into three groups: nanoclusters (less than 2 nm in diameter), nanoparticles (NPs) (2-100 nm in diameter) and nanocrystals (more than 100 nm) (see, for example, Lu, Y. et al., Gold Clusters, Colloids and Nanoparticles I. Springer International Publishing, 2013. 117-153; Mingos, D. M. P., Gold Clusters, Colloids and Nanoparticles I. Springer International Publishing, 2014. 1-47; Dreaden, E. C. et al., Chemical Society Reviews 41.7 (2012): 2740-2779; Boisselier, E. et al., Chemical society reviews 38.6 (2009): 1759-1782; Fran£ois, A. et al., ChemMedChem 6.11 (2011): 2003-2008; Muddineti, O. S. et al., International journal of pharmaceutics 484.1 (2015): 252-267; and Niikura, K. et al., ACS applied materials & interfaces 5.9 (2013): 3900-3907, all incorporated by reference in their entirety for all purposes).

The metallic surface can be functionalized with different chemical groups such as thiol groups leading to a very efficient stabilization of the gold core. However, in this context, there are still a number of challenges related to the stability of AuNPs in different conditions, as different buffers, times and temperatures. Indeed, for future medical applications, AuNPs must be ultrastable to remain biocompatible and not lead to aggregation or toxicity.

For example, Gupta, A. et al synthesized AuNPs functionalized with ethylene glycol and a zwitterionic group (see Gupta, A. et al ACS Applied Materials & Interfaces 8.22 (2016): 14096-14101, incorporated by reference in its entirety for all purposes). These NPs were stable to pH changes, salt concentrations, heating and freeze-drying. As another example, Tatumi and Fujihara synthesized AuNPs functionalized with a zwitterionic liquid which were stable in solutions that contain high concentrations of salts and proteins (see Tatumi, R. et al., Chemical Communications 1 (2005): 83-85, incorporated by reference in its entirety for all purposes). However, the use of these AuNPs might be complicated in vivo due to their potential toxicity arising from their zwitterionic groups leading to the presence of charged groups in periphery (see Fröhlich, E. International journal of nanomedicine 7 (2012): 5577-5591, incorporated by reference in its entirety for all purposes). Zhou et al synthesized AuNPs with a one-step process in water by adding a reducing agent to a mixture of gold and polyvinylpyrrolidone (PVP) (see Zhou, M. et al., Nanotechnology 20.50 (2009): 505606, incorporated by reference in its entirety for all purposes). The stabilizing groups were carboxyl groups, where a transfer of the oxygen electron to the gold helped to stabilize the NPs in water media with high concentrations of salts. However, the use of thiols groups on the gold core leads to a better stability and should thus be used for in vivo applications (see Dreaden, E. C. et al., Chemical Society Reviews 41.7 (2012): 2740-2779; Kumara, C. et al., Gold Clusters, Colloids and Nanoparticles I. Springer International Publishing, 2014. 155-187; and Brust, M. et al., Journal of the Chemical Society, Chemical Communications 7 (1994): 801-802, all incorporated by reference in their entirety for all purposes).

Several synthesis methods lead to spherical AuNPs. Brust and Turkevish methods are among the most used (see Brust, M. et al. Journal of the Chemical Society, Chemical Communications 7 (1994): 801-802; Turkevich, J. et al., Journal of colloid Science 9 (1954): 26-35; Yang, X. et al., Chemical reviews 115.19 (2015): 10410-10488, all incorporated by reference in their entirety for all purposes). The Brust method is a single step synthesis and generally leads to smaller and more stable NPs than the citrate ones. However, the Brust method involves the use of toxic reagents as toluene and tetraoctylammonium bromide (see Mingos, D. M. P., Gold Clusters, Colloids and Nanoparticles I. Springer International Publishing, 2014. 1-47; Dreaden, E. C. et al., Chemical Society Reviews 41.7 (2012): 2740-2779; and Brust, M. et al. Journal of the Chemical Society, Chemical Communications 7 (1994): 801-802, all incorporated by reference in their entirety for all purposes). Although synthesis of AuNPs could be achieved directly in water, this method was found to form few stable NPs and only worked with ligands that could quickly stabilize the gold core (low molecular weight ligands).

The present invention thus proposes new synthesis conditions leading to ultrastable biocompatible AuNPs.

### SUMMARY OF THE INVENTION

The invention therefore provides ultrastable nanoparticles (AuNPs) comprising biocompatible ligand. In an aspect, the invention describes use of AuNPs as a carrier for a medicament against a disorder or disease.

In a first aspect of the invention, there is provided AuNPs coated with a biocompatible ligand, wherein said NPs are stable after cold or heat treatment. Particularly, the AuNPs of the present invention, are ultrastable in that said NPs have a defined absorption spectrum before the cold or heat treatment, and the absorption spectrum is substantially unchanged after the treatment.

In another aspect of the invention, there is provided AuNPs coated with a biocompatible ligand, wherein said biocompatible ligand is devoid of the presence of any charged groups in periphery.

In a further aspect, the invention describes a composition comprising AuNPs as defined herein, in combination with a physiologically-acceptable excipient.

In a further aspect, the invention describes a formulation for the mucosal treatment of a disease, comprising the composition as defined herein, in combination with a drug effective for the treatment of said disease.

In a further aspect, the invention describes a method for the treatment of a disease or disorder comprising a mucosal-directed administration of a drug, said method comprising administering a therapeutic dose of the formulation as defined herein.

In a further aspect, the invention describes a method for the treatment of glaucoma comprising the step of: topically administering to a cornea, a therapeutically-effective dose of travoprost formulated with AuNP as defined herein.

In a further aspect, the invention describes a method for the synthesis of AuNPs, said method comprising the steps of: dissolving chloroauric acid (HAuCl₄) in an acetonitrile-containing solvent to form a partially-reduced gold solution; dissolving biocompatible ligand-SH in an organic solvent; adding said dissolved biocompatible ligand-SH to said partially-reduced gold solution to form a partially-reduced mixture; reducing the partially-reduced mixture to form a reduced mixture; and leaving said reduced mixture for sufficient time to proceed to nucleation and crystallization.

### DETAILED DESCRIPTION OF THE INVENTION

### Description of the Figures

FIG. 1. Synthesis and reduction of AuNPs.

FIG. 2. UV-visible spectrum of AuNPs capped with PEG 2000-SH.

FIG. 3. UV-visible spectra of AuNPs capped with PEG 2000-SH after freeze-drying (or lyophilization) and heating over night (16 hours) at 65° C.

FIG. 4. UV-visible spectra of AuNPs capped with (A) PEG 800-SH, (B) PEG 2000-SH and (C) PEG 6000-SH after three consecutive cycles of heating over night (16 hours) at 65° C. (grey lines are the initial spectra with AuNPs at a concentration of 0.03 mg/mL) (black lines are the spectra recorded after three heating cycles).

FIG. 5. UV-visible spectra of AuNPs capped with (A) PEG 800-SH, (B) PEG 2000-SH and (C) PEG 6000-SH after three successive cycles of freeze-drying (or lyophilization) (grey lines are the initial spectra with AuNPs at a concentration of 0.04 mg/mL) (black lines are the spectra recorded after three freeze-drying cycles).

FIG. 6. (A) Transmission electron microscopy (TEM) images of AuNPs capped with PEG 2000-SH (scale bar =20 nm). (B) Size distribution of the AuNPs.

FIG. 7. UV-visible spectra of AuNPs alone (bottom spectrum) and in the presence of mucins (top spectrum).

FIG. 8. Fluorescence spectra of mucins alone (top spectrum) and in the presence of AuNPs (bottom spectrum).

FIG. 9. Kinetics of encapsulation of travoprost by UV spectroscopy at 278 nm.

FIG. 10. Kinetics of encapsulation of timolol by UV spectroscopy at 295 nm.

FIG. 11. Illustration of the number of magnetizations required to remove a sufficient number of nanoparticles. For instance, the graph illustrates that three magnetizations are required to remove 98% of the nanoparticles. The residual absorbance is then comparable to the limit of the error of the apparatus.

FIG. 12. Calibration curve of travoprost by UV-visible spectroscopy in PBS-T (0.0005% tween).

FIG. 13. Calibration curve of travoprost by high performance liquid chromatography (HPLC) in PBS-T (0.0005% tween).

FIG. 14. Calibration curve of timolol by UV-visible spectroscopy in PBS-T (0.0005% tween).

FIG. 15. Percentage of encapsulated travoprost. This experiment was performed with initial concentrations of 80 μM and 120 μM of travoprost with 69 nM of AuNPs.

FIG. 16. UV-visible spectra of different concentrations of travoprost encapsulated into AuNPs. The first travoprost's peak (diamond line) and the second travoprost's peak (square line) are both shown.

FIG. 17. Model of glaucoma induction in mice. (A) Injection of polystyrene microbeads (10 μm) in the anterior chamber of the mouse eye blocked the aqueous humor drainage and induced intraocular pressure (IOP) elevation. (B) Measurement of the IOP with a tonometer (TonoLab) before microbead injections and 10 days after. Each dot represents values from single animals. Statistics: P<0.001, t-test.

FIG. 18. The effects of travoprost encapsulated in AuNPs on the IOP of glaucomatous mice. (A) Changes in IOP after topical application of travoprost. (B) Changes in IOP after topical application of travoprost combined to AuNPs. Mean IOP±standard error of the mean (S.E.M.) was calculated in 9 glaucomatous mice. (C) The mean IOP is presented for the same mice as in (A) and (B) before treatment and 24 h after. Statistics: p<0.05, t-test.

### ABBREVIATIONS AND DEFINITIONS

### Definitions

The term “about” or “substantially” as used herein refers to a margin of + or −10% of the number indicated. For sake of precision, the term about when used in conjunction with, for example: 90% means 90%+/−9% i.e. from 81% to 99%. More precisely, the term about refers to + or −5% of the number indicated, where for example: 90% means 90%+/−4.5% i.e. from 86.5% to 94.5%.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

### DETAILED DESCRIPTION OF PARTICULAR EMBODIMENTS

Herein presented is novel synthesis conditions leading to ultrastable biocompatible AuNPs. In contrast to the Brust methodology, the present proposed conditions include the use of acetonitrile in a one-step procedure without the use of any stabilizer.

### Method of Synthesis

According to a particular embodiment, the invention describes a method for the synthesis of AuNPs, said method comprising the steps of: dissolving chloroauric acid (HAuCl₄) in an acetonitrile-containing solvent to form a partially-reduced gold solution; dissolving biocompatible ligand-SH in an organic solvent; adding said dissolved biocompatible ligand-SH to said partially-reduced gold solution to form a partially-reduced mixture; reducing the partially-reduced mixture to form a reduced mixture; and leaving said reduced mixture for sufficient time to proceed to nucleation and crystallization.

According to a particular embodiment, the method further comprises the step of: evaporating said acetonitrile solvent mixture and organic solvent from said second mixture to obtain a solution of AuNPs. More particularly, the method further comprises the step of: submitting said aqueous AuNPs solution to dialysis to remove excess ligand.

According to a particular embodiment, there is provided a method for the synthesis of AuNPs, said method comprising the steps of: providing chloroauric acid (HAuCl₄) dissolved in a mix acetonitrile/isopropyl alcohol to form a gold solution; providing PEG 2000-SH ligand dissolved in isopropyl alcohol; adding said dissolved PEG 2000-SH to said gold solution to form a first mixture; agitating said first mixture; adding dropwise sodium borohydride (NaBH₄) dissolved in water to the first mixture under intense agitation to form a second mixture; leaving said second mixture for sufficient time to stabilize; evaporating said acetonitrile/isopropyl alcohol from said second mixture to obtain an aqueous solution of AuNPs; and submitting said aqueous AuNPs solution to dialysis to remove excess PEG-2000.

According to another particular embodiment, there is provided a method for the synthesis of AuNPs, said method comprising the steps of: providing HAuCl₄ dissolved in a mix acetonitrile/isopropyl alcohol to form a gold solution; providing PEG 800-SH ligand dissolved in isopropyl alcohol; adding said dissolved PEG 800-SH to said gold solution to form a first mixture; agitating said first mixture; adding dropwise NaBH₄ dissolved in water to the first mixture under intense agitation to form a second mixture; leaving said second mixture for sufficient time to stabilize; evaporating said acetonitrile/isopropyl alcohol from said second mixture to obtain an aqueous solution of AuNPs; and submitting said aqueous AuNPs solution to dialysis to remove excess PEG-800.

According to a further particular embodiment, there is provided a method for the synthesis of AuNPs, said method comprising the steps of: providing HAuCl₄ dissolved in a mix acetonitrile/isopropyl alcohol to form a gold solution; providing PEG 6000-SH ligand dissolved in isopropyl alcohol; adding said dissolved PEG 6000-SH to said gold solution to form a first mixture; agitating said first mixture; adding dropwise NaBH₄ dissolved in water to the first mixture under intense agitation to form a second mixture; leaving said second mixture for sufficient time to stabilize; evaporating said acetonitrile/isopropyl alcohol from said second mixture to obtain an aqueous solution of AuNPs; and submitting said aqueous AuNPs solution to dialysis to remove excess PEG-6000.

### Ultrastable NPs

According to a particular embodiment, the herein presented synthesis yields in ultrastable AuNPs coated with a biocompatible ligand, wherein the NPs are stable after cold or heat treatment. Particularly, the NPs are stable under cold and heat treatment.

More particularly, the AuNPs have a defined absorption spectrum before the treatment, and the absorption spectrum is substantially unchanged after the treatment.

According to a particular embodiment, the AuNPs have an absorption spectrum peak at about 515 nm, and the absorption spectrum peak is substantially unchanged after cold or heat treatment. Particularly, the cold treatment may comprise at least one cycle of freeze-drying. Alternatively, the heat treatment comprises at least one cycle of heating overnight.

According to a particular embodiment, the ultrastable AuNPs present no aggregation or no precipitation after said freeze-drying or heating cycles. Particularly, the ultrastable AuNPs present no aggregation and no precipitation after said freeze-drying and heating cycles. More particularly, the ultrastable AuNPs are soluble in water, before and after heat and/or cold treatment.

According to a particular embodiment, the AuNPs are coated with a biocompatible ligand that is devoid of any charge groups in periphery. More particularly, the AuNPs are coated with a biocompatible ligand that is devoid of zwitterionic groups leading to the presence of charged groups in periphery.

According to a particular embodiment, the ultrastable AuNPs have a core diameter ranging from 1.0 to 100 nm, particularly ranging from 1.0 to 10 nm, more particularly from 1.0 to 5 nm, still more particularly from 1.9 to 2.4, most particularly ranging from about 1.88 to 2.37 nm. Particularly, the types of ultrastable NPs of the invention may be chosen from: nanospheres, nanorods, nanocubes, nanoprisms, nanoblocks, nanotetrapods, nanomultipods, nanostar and branched NPs.

### Biocompatible Ligand

In accordance with a particular embodiment, the biocompatible ligand is muco-adhesive. Alternatively, the biocompatible ligand is muco-penetrating.

According to a particular embodiment, the biocompatible ligand is selected from the group consisting of: polyethylene glycol (PEG), chitosan, polyvinyl pyrrolidone, polyvinyl alcohol, polyacrylic acid, polyacrylamide, N-(2-hydroxypropyl) methacrylamide, divinyl ether-maleic anhydride, polyoxazoline, polyphosphates, polyphosphazenes, xanthan, pectins, dextran, carrageenan, guar gum, cellulose ethers, hyaluronic acid, albumin and starch based derivatives.

Particularly, the ligand comprises PEG, more particularly, thiolated PEG. Most particularly, the thiolated PEG has a molecular weight from about 10,000 g/mol to about 500 g/mol, more particularly from about 8000 to about 800 g/mol.

Most particularly, the chosen ligand for the stabilization of the AuNPs may be thiolated polyethylene glycol groups (PEG 2000-SH) which has a molecular weight of about 2000 g/mol.

Alternatively, the chosen ligand for the stabilization of the AuNPs may be thiolated polyethylene glycol groups (PEG 800-SH) which has a molecular weight of about 800 g/mol.

Alternatively, the chosen ligand for the stabilization of the AuNPs may be thiolated polyethylene glycol groups (PEG 6000-SH) which has a molecular weight of about 6000 g/mol.

### Use as Carrier

In accordance with a particular embodiment of the invention, there is provided a use of AuNPs as defined herein as a carrier for a medicament or a drug against a disorder or disease. Particularly, the disease or disorder is a disorder or disease of a mucous membrane.

Particularly, the medicament or drug is carried by the AuNPs to a site of administration, most particularly a mucous membrane, preferably said mucous membrane is selected from the group consisting of: eye mucosa, bronchial mucosa, endometrium, esophageal mucosa, gastric mucosa, intestinal mucosa, nasal mucosa, oral mucosa, penile mucosa, vagina mucosa and anal mucosa. For example, the mucous membrane is ocular or ophthalmic mucous membrane.

### Composition, Formulation

In accordance with a particular embodiment, the present invention provides a composition comprising AuNPs as defined herein, in combination with a physiologically-acceptable excipient.

According to a particular embodiment, there is provided a formulation for the mucosal treatment of a disease, comprising the composition as defined herein, in combination with a drug effective for the treatment of the disease.

### Method of Treatment

In accordance with a particular embodiment, there is provided a method for the treatment of a disease or disorder comprising a mucosal-directed administration of a drug, this method comprising administering a therapeutic dose of the composition or formulation as defined herein. In particular, the invention provides a method for the treatment of glaucoma comprising the step of administering an anti-blood pressure active concentration of an anti-glaucoma drug combined with the AuNPs of the present invention.

### Therapeutic Indications

In accordance with a further aspect of the invention, there is described a method for the treatment of a disease or disorder comprising a mucosal-directed administration of a drug, said method comprising administering a therapeutic dose of the composition or formulation as defined herein. According to a particular aspect, the disease or disorder an ophthalmic disease such as: glaucoma, macular degeneration, blepharitis, conjunctivitis, dry eye syndrome, eye allergies, and eye infections.

In particular, when the disease to be treated is glaucoma, the medicament or drug is selected from the group consisting of: travoprost; pilocarpine, carbachol, latanoprost, bimatoprost, tafluprost, timolol, betaxolol, carteolol, levobunolol, apraclonidine, brimonidine, dorzolamide, brinzolamide, acetazolamide and methazolamide; particularly timolol and most particularly travoprost.

### Mode of Administration

According to a further aspect, the AuNPs, formulation, medicament, or combination, are formulated for relevant administration for the particular mucous membrane. Most particularly, the AuNPs, formulation, medicament, or combination are adapted to be delivered to the mucous membrane via topical, oral or aerosol administration. Most particularly, the AuNPs, formulation, medicament, or combination, are formulated for topical administration on the ocular or ophthalmic mucous membrane.

### Dosage

According to a particular aspect, the invention provides a method for the treatment of glaucoma comprising the step of: topically administering to a cornea, a dose of at least about 0.004% of travoprost formulated with AuNPs as defined herein. Particularly, the formulated drug is administered at intervals of less than once per day, most particularly, no more often than every other day (i.e. once per two days).

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### EXAMPLES

### Example 1

### Methods

### Chemical Synthesis

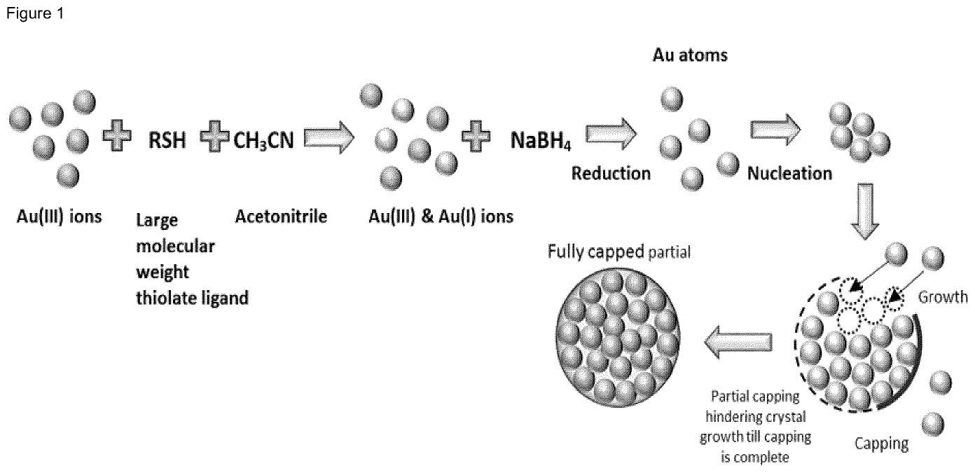

First, 0.0635 mmol of chloroauric acid (HAuCl₄) was dissolved in 15 mL of a mix acetonitrile/isopropyl alcohol (50/50) (FIG. 1). Then, PEG 2000-SH (0.026 g, 0.013 mmol) was dissolved in 10 mL of isopropyl alcohol, and was then added to the gold solution. The molar ratio of gold to PEG used was 4.9:1. The mixture was then left under agitation for one hour. 0.028 g of NaBH₄ in 10 mL of ice cold water was then added to the mixture dropwise at a rate of 1.3 mL/min with intense agitation (1200 rpm). After complete addition of the NaBH₄ the mixture was left to stabilize for three more hours. The solvent was then evaporated with a rotary evaporator until a volume of 5 mL was obtained. The AuNPs solution was then loaded in a dialysis tube for two days with several washings in order to remove the excess ligands. The AuNPs were then stored in an amber colored glass vial.

The same chemical synthesis was used replacing PEG 2000-SH by PEG-800-SH or PEG-6000-SH.

### Physicochemical Characterizations

The weight of AuNPs was determined with a microbalance. Their absorbance was determined by UV-visible spectroscopy. The stability of NPs was also assessed after freeze-drying (samples frozen at −80° C. for 15 min then freeze-dried for 3 hours), and overnight (16 hours) heating at 65° C. by comparing their UV spectra before and after these treatments. The diameter of the gold core was determined using Transmission Electron Microscopy (TEM). This diameter was then used to calculate the number of gold atoms using the equations below.

$$\begin{matrix}Volume of \text{?} \text{?} - \frac{\text{?} π \text{?} {( radius in \text{?} )}^{3}}{3} & ( equation 1 ) \\ Number \text{?} \text{?} \text{?} the goal core - \frac{volume of the core in \text{?}}{17 \text{?}} \\ \text{?} \text{indicates text missing or illegible when filed} & ( equation 2 )\end{matrix}$$

Mucoadhesion measurements were performed by UV-visible and fluorescence spectroscopies. Fluorescence intensities were corrected for the inner filter effect, taking both excitation and emission into account. (see Coutinho, A. et al., J. Chem. Educ 70.5 (1993): 425; and Komarnicka, U. et al., Dalton Transactions 45.12 (2016): 5052-5063, both incorporated by reference in their entirety for all purposes). First, the excitation light used to illuminate the fluorophore, f, can also be absorbed by another component, i, present in the sample. Given that the total absorbance (Absₜ) is given by Absₜ=Absᵢ+Abs_{f}, the correction factor (C1) at the excitation wavelength is given by the following equation:

$$C 1 - \frac{\text{?}}{\text{?}} ( \frac{1 - 10 \text{?}}{1 - {10}^{-} \text{?}} ) \\ \text{?} \text{indicates text missing or illegible when filed} \qquad ( equation 3 )$$

Also, fluorescence emission may be re-absorbed by another component, e, present in the sample. In this case, the correction factor (C2) at the emission wavelength is given by the following equation:

$$C 2 = \frac{2.303 \text{?}}{1 - {10}^{-} \text{?}} \\ \text{?} \text{indicates text missing or illegible when filed} \qquad ( equation 4 )$$

### Results

The initial mixing of HAuCl₄ and PEG 2000-SH resulted in a partial reduction of gold. The complete reduction was then achieved with the addition of NaBH₄ which initiated the crystal formation. The gold atoms were then capped and stabilized with the thiolated ligands. The speed of crystal growth versus ligand capping determined the success of this synthesis. The presence of acetonitrile in the initial reaction slowed the growth of crystals and allowed better capping and stabilization.

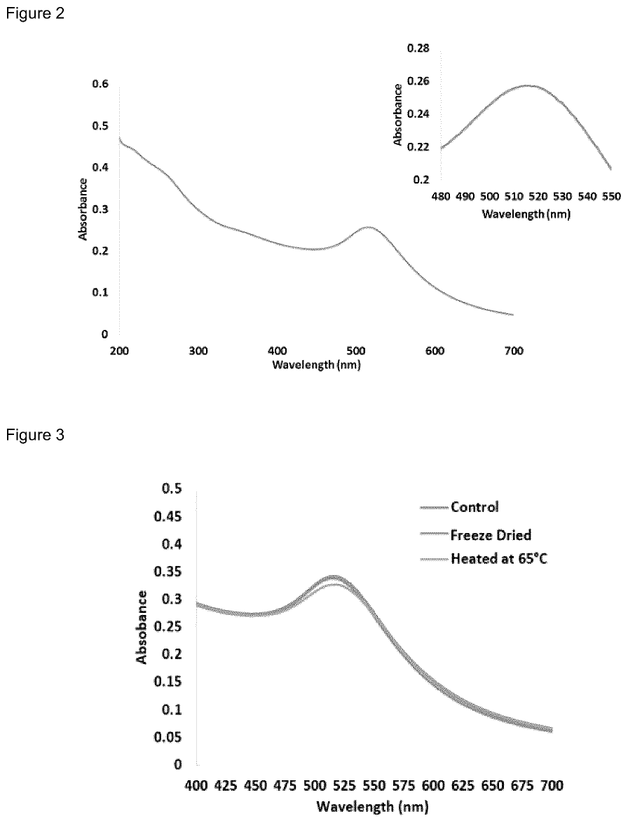

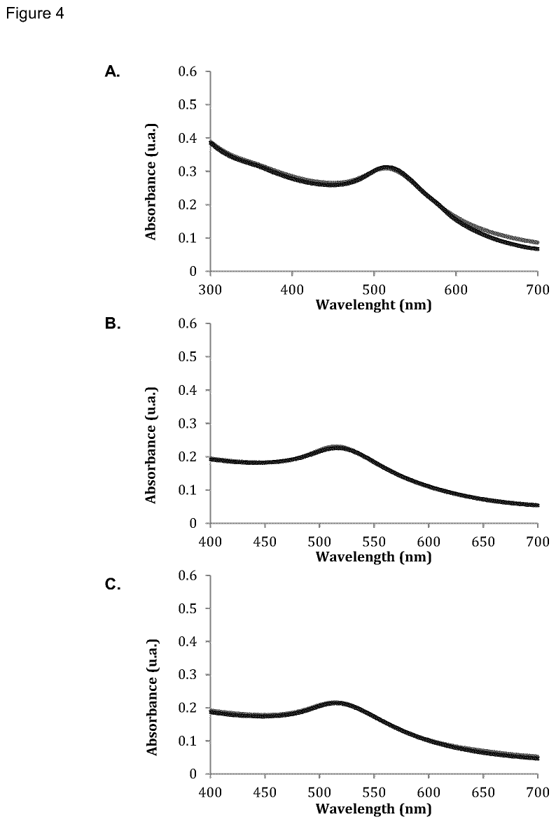

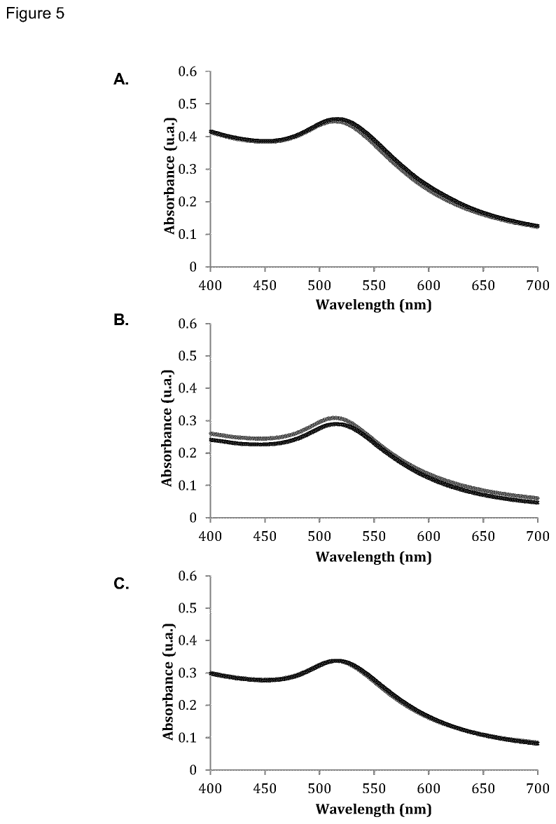

These new experimental conditions led to ultrastable NPs with an absorption spectrum of AuNPs around 515 nm (FIG. 2). This spectrum did not evolve after one cycle of freeze-drying (FIG. 3, orange spectrum). After heating, the spectrum slightly evolved but no aggregation or precipitation was observed (FIG. 3, grey spectrum). Indeed, the heating could have slightly modified the arrangement of the ligands onto the gold core which could have influenced the NPs absorption. After these treatments, the AuNPs were still soluble and stable in water. These results were repeated with other types of thiolated polyethylene glycol groups (PEG 800-SH or PEG-6000) and with three repeated cycles of either freeze-drying or heating cycles. FIG. 4 displays the UV-visible spectra of AuNPs capped with (A) PEG 800-SH, (B) PEG 2000-SH and (C) PEG 6000-SH after three repetitive cycles of heating overnight (16 hours) at 65° C. Again, we can see that the spectrum did not significantly evolve (FIGS. 4 (A), (B) and (C), purple spectra). FIG. 5 displays UV-visible spectra of AuNPs capped with (A) PEG 800-SH, (B) PEG 2000-SH and (C) PEG 6000-SH after three cycles of freeze-drying or (lyophilization). Again, the spectrum did not significantly evolve (FIGS. 5 (A), (B) and (C), purple spectra) after the three cycles.

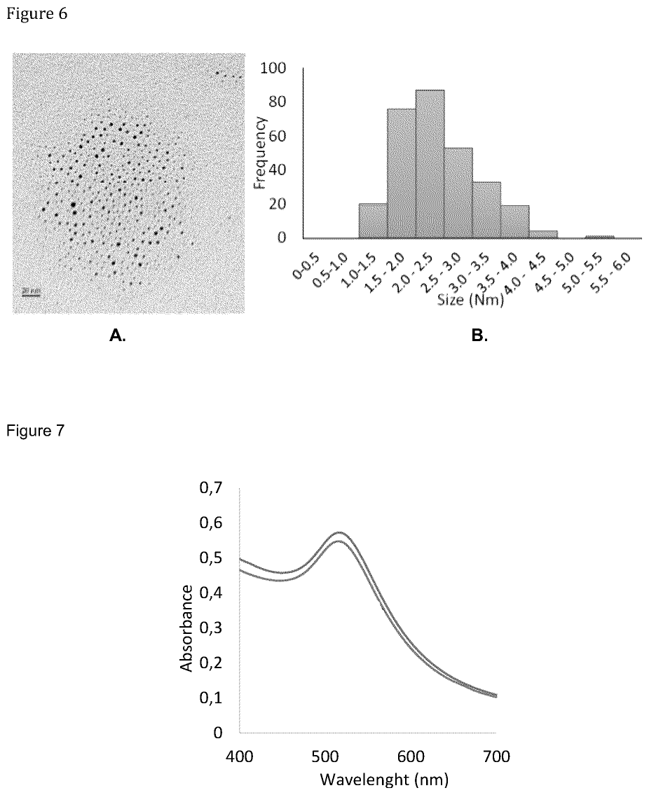

TEM images revealed spherical particles with a mean diameter of 2.1±0.7 nm (FIG. 6) for the AuNPs PEG 2000-SH, and respectively of 1.88±0.69 nm and 2.37±1.03 nm for the AuNPs PEG 800-SH and PEG 6000-SH. Moreover, according to the equations described in the methods section, the average number of gold atoms in the core was found to be 309. Elemental analysis (fire assay and induction assay) were performed by SRC Geoanalytical Laboratories (Saskatchewan). The resulting molecular weight of the AuNPs capped with PEG 2000-SH was found to be 4 326 893.88 g/mol.

The Zeta potential analysis technique was performed to determine the surface charge of the AuNPs. The Zeta potential is one of the fundamental parameters known to affect stability. The Zeta potential obtained for the AuNPs PEG 800-SH, PEG 2000-SH, and PEG 6000-SH were respectively (−7.05±7.46 mV), (−8.02±4.28 mV) and (−1.79±4.28 mV).

The Dynamic Light Scattering (DLS) technique was used to calculate the hydrodynamic diameter of the AuNPs. The hydrodynamic diameter potential obtained for the AuNPs PEG 800-SH, PEG 2000-SH, and PEG 6000-SH were respectively (33±1 nm), (31±4 nm) and (54±2 nm).

Mucoadhesion measurements were performed with UV and fluorescence spectroscopies. The slight increase of the absorbance intensity of the AuNPs observed by UV spectroscopy illustrated the perturbation of the metallic core by the mucins, and thus their interaction (FIG. 7).

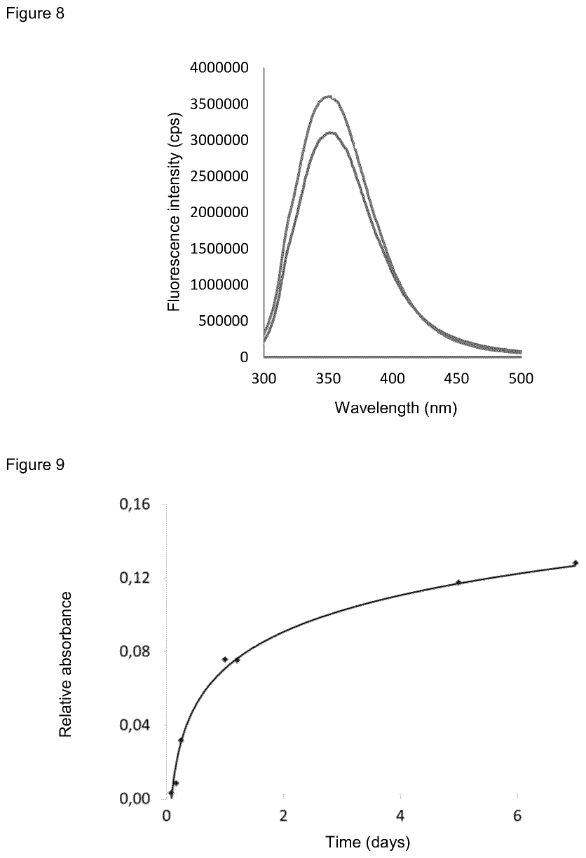

The mucoadhesion between the AuNPs and the mucins could also be illustrated by the quenching of fluorescence of mucins (FIG. 8). Indeed, after the two correction factors application as explained in the above section entitled “Physicochemical characterizations”, the decrease in fluorescence intensity of mucins in the presence of AuNPs was due to their interaction, and thus to the mucoadhesive properties of the AuNPs.

### Conclusions and Perspectives

For medical applications, AuNPs must be ultrastable to increase their efficiency and decrease their toxicity. The new experimental conditions presented herein led to ultrastable AuNPs, even after freeze-drying and heating. These new ultrastable AuNPs have a promising future in the drug delivery area.

### Example 2

### Encapsulation Protocol

### Conditions of Encapsulation

The AuNPs and the drug are brought into contact in a screw-cap 2 mL microtube, with magnetic bar stirring, at 37° C. for a time determined according to the kinetics of encapsulation obtained by UV-visible spectroscopy.

### Encapsulation of Travoprost

In the case of travoprost, an encapsulation time of five days was chosen according to the encapsulation kinetics determined by UV spectroscopy at 278 nm (FIG. 9).

### Encapsulation of Timolol

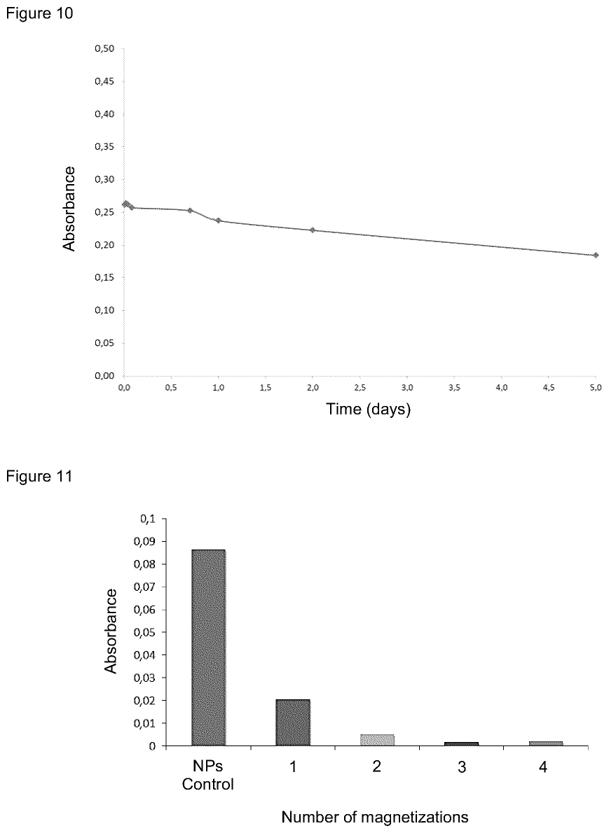

In the case of timolol, an encapsulation time of 30 minutes was chosen according to the encapsulation kinetics determined by UV spectroscopy at 295 nm (FIG. 10).

### Protocol to Determine the Encapsulation Efficiency

The method used to determine the encapsulation efficiency rely on an indirect assay by evaluating what has not been encapsulated. Once the encapsulation is complete, the entire mixture is brought into contact with magnetic beads to allow the nanoparticles that contain the encapsulated drug to be removed from the medium. The residual supernatant is then analyzed by UV-visible spectroscopy and high-performance liquid chromatography (HPLC).

To do so, a solution of 0.0005% phosphate buffered saline Tween™ (PBS-T) was prepared. The magnetic beads were then cleaned and 100 μL of magnetic beads were placed in a 1.5 mL microtube. The beads were then magnetized, and the supernatant was removed.

1 mL of the prepared PBS-T was added to the beads, the beads were then suspended with a vigorous vortex for 30 seconds. The beads were then magnetized, and the supernatant was removed. These steps were repeated consecutively three times. 4 μg of antibody were then added in a final volume of 200 μl and the beads were suspended again. The suspension was stirred for 10 minutes using the thermomixer (1000 rpm, at a temperature of 21° C.). The beads were then once again magnetized, and the supernatant was removed.

Another 1 mL of the prepared PBS-T was added to the beads. The beads were then again suspended with a vigorous vortex for 30 seconds. The beads were then once again magnetized, and the supernatant was removed. These steps were also repeated successively three times.

0.5 mL of the sample for which you want to assay the encapsulation was then added. The solution was stirred for 3 hours using the thermomixer (1000 rpm, at a temperature of 21° C.). The beads were then once again magnetized, and the supernatant was recovered.

The recovered supernatant was then put back in the microtube, the beads were then re-magnetized, and the supernatant was recovered. These steps were performed twice.

The final supernatant was analyzed by UV-Visible spectroscopy (λ=200-700 nm) and by high-performance liquid chromatography (HPLC).

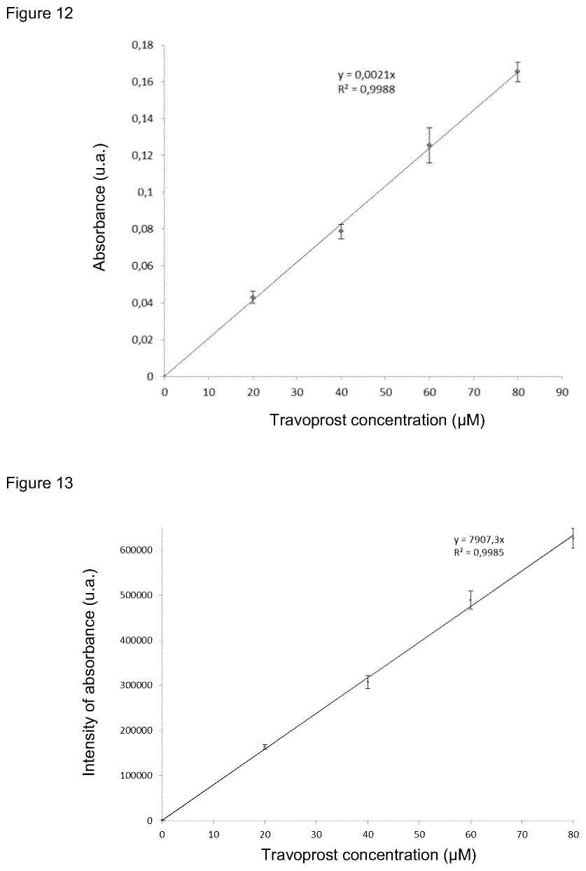

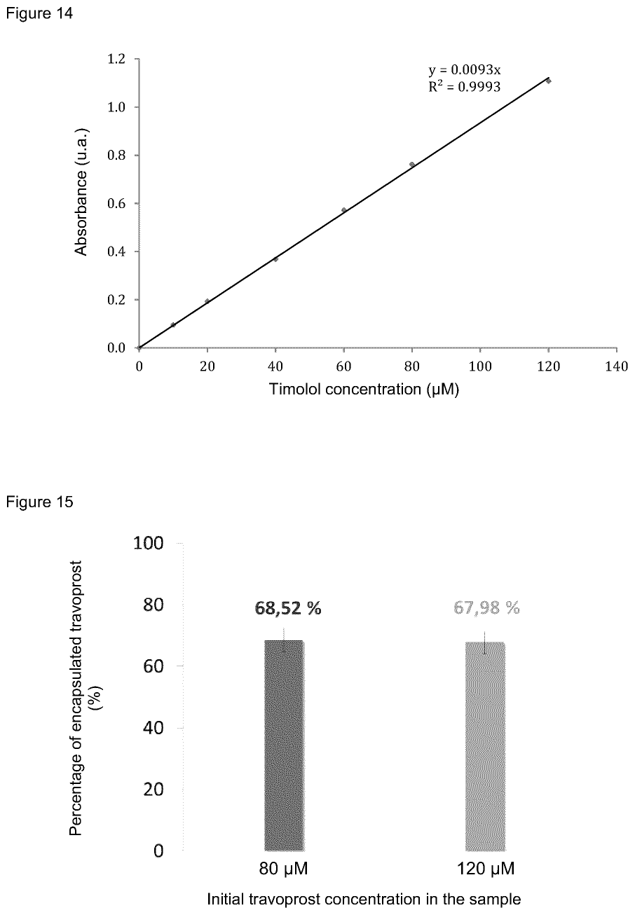

FIG. 11 illustrates that three magnetizations were required to remove 98% of the nanoparticles. The residual absorbance was then comparable to the limit of the error of the apparatus. Calibration curves of travoprost by UV-visible spectroscopy (FIG. 12) and by high performance liquid chromatography (HPLC) (FIG. 13) in PBS-T (0.0005% tween) were performed. The calibration curve of timolol by UV-visible spectroscopy in PBS-T (0.0005% tween) was also performed (FIG. 14).

FIG. 15 illustrates the percentage of encapsulated travoprost, this experiment was performed with an initial concentration of travoprost in the sample of 80 μM and 120 μM and a concentration of AuNPs of 69 nM. The obtained percentages were about 68.52%±4.032% of encapsulated travoprost for an initial 80 μM and about 67.98%±3.877% of encapsulated travoprost for an initial 120 μM.

### Example 3

In Vivo Results Obtained with the Ultrastable AuNPs

### Material and Methods

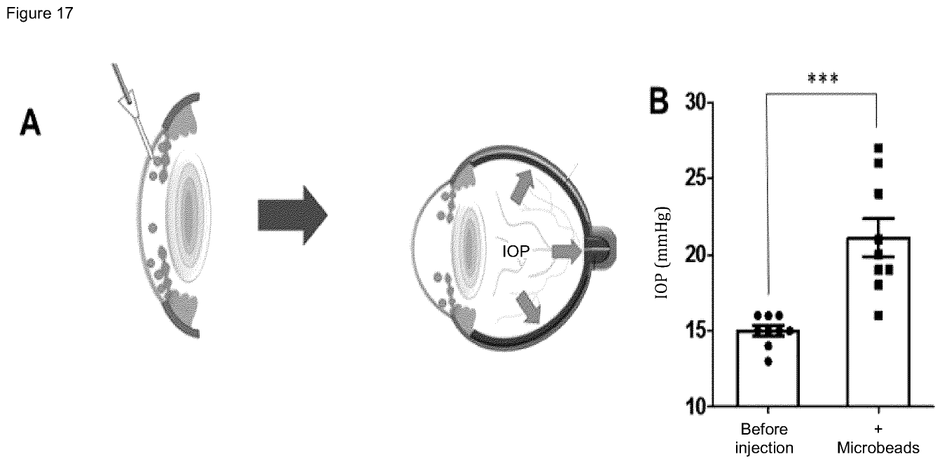

An in vivo glaucoma model involving the injection of exogenous microbeads into the anterior chamber was used (FIG. 17A). The injected beads partially block the trabecular meshwork resulting in intraocular pressure (IOP) elevation while causing no damage to the site of drug action. Two-month-old C57BL/6J mice were used. The IOP was followed with a tonometer (Tonolab®) specially designed for rodents. Animals were injected with 5 μL of microbeads with a dimeter of about 10 μm (7.2×10⁶ microbeads/mL) in the anterior chamber of the left eye. For the encapsulation experiments, travoprost was solubilized in water/ethanol (50/50) in the presence of AuNPs. The solution was heated (65° C.) in order to evaporate the ethanol and to induce conformational changes of PEG molecules leading to a better encapsulation. The solution was thus completed with water until the initial volume.

### Results

### Drug Vector Toxicity

The AuNPs (77 μmol) were administered topically to the mouse cornea and no external sign of irritation or ocular inflammation appeared.

### Encapsulation of Travoprost

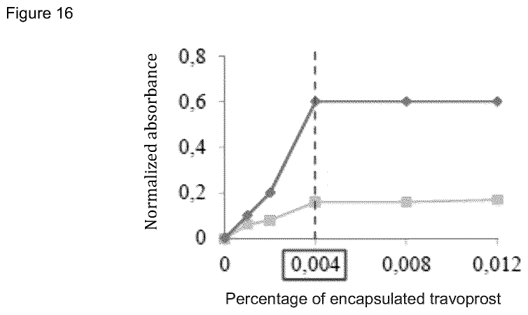

The characterization of the encapsulation was carried out by UV-visible spectroscopy. Using this characterization technique, it was possible to detect the absorption of travoprost in an aqueous medium demonstrating its solubility via its encapsulation within AuNPs. Moreover, the minimum concentration of AuNPs that encapsulates 0.004% of travoprost, the dose currently used in the market (FIG. 16) was defined. Furthermore, the maximum of the plasmon band of the AuNPs shifted by 20 nm during the titration in water, highlighting the presence of travoprost close to the heart of the AuNPs in the cavities formed by PEGs. These results confirm the ability of AuNPs to effectively encapsulate hydrophobic active molecules.

### Efficiency of the New Gold-Travoprost NPs Medication

### Validation of the Model of Hypertensive Glaucoma in Mice.

Hypertensive glaucoma was induced in adult C57BL/6 mice by injecting 5 μL of polystyrene microbeads (35,000 microbeads) with an average diameter of 10 μm in the anterior chamber of the eye. The dispersion of the microbeads at the level of the iridocorneal angle blocks the passage of the aqueous humor towards the trabecular meshwork (FIG. 17A). Mechanically, the decrease in drainage of the aqueous humor produces an increase in intraocular pressure which is measured with a tonometer of the Tonolab brand. A very significant increase in intraocular pressure was observed ten days after the injection of microbeads in a group of 15 glaucomatous mice (FIG. 17B)

### Improvement by AuNPs of the Effects of Travoprost on Intraocular Pressure in Glaucomatous Mice.

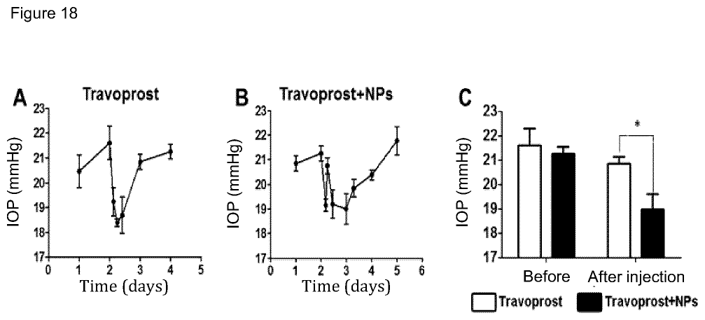

A comparative experiment was conducted with travoprost with or without AuNPs in glaucomatous mice. Travoprost without AuNPs significantly but transiently decreased intraocular pressure in glaucomatous mice (FIG. 18A); the level of intraocular pressure returned to a high level, similar to that before treatment, after 24 hours. Comparatively, combined treatment of travoprost and AuNPs had a more sustained effect on intraocular pressure, the level of which remained significantly lower after two days (FIGS. 18 B and C). These results demonstrate that these new ultrastable AuNPs are able to double the duration of action of travoprost (length of time that the travoprost is effective) on intraocular pressure, after a single application.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

All patents, patent applications and publications mentioned in this specification are herein incorporated by reference to the same extent as if each independent patent, patent application or publication was specifically and individually indicated to be incorporated by reference.

1. Gold nanoparticles (AuNPs) coated with a biocompatible ligand, wherein said nanoparticles have a periphery and are stable after cold or heat treatment, wherein said biocompatible ligand is devoid of a charged group in said periphery.

2. The gold nanoparticles of claim 1, wherein said gold nanoparticles have a defined absorption spectrum before said treatment, and said absorption spectrum is substantially unchanged after said treatment.

3. (canceled)

4. The gold nanoparticles of claim 1, wherein said biocompatible ligand is muco-adhesive.

5. The gold nanoparticles of claim 1, wherein said biocompatible ligand is muco-penetrating.

6. The gold nanoparticles of claims 1, wherein said biocompatible ligand is selected from the group consisting of: polyethylene glycol (PEG), chitosan, polyvinyl pyrrolidone, polyvinyl alcohol, polyacrylic acid, polyacrylamide, N-(2-hydroxypropyl) methacrylamide, divinyl ether-maleic anhydride, polyoxazoline, polyphosphates, polyphosphazenes, xanthan, pectins, dextran, carrageenan, guar gum, cellulose ethers, hyaluronic acid, albumin and starch based derivatives.

7-13. (canceled)

14. The gold nanoparticles of claim 1, wherein said gold nanoparticles present no aggregation or no precipitation after said at least one freeze-drying cycle or at least one overnight heating.

15. (canceled)

16. The gold nanoparticles of claim 1, wherein said gold nanoparticles have a core diameter and wherein said core diameter ranges from 1.0 to 100 nm.

17-21. (canceled)

22. A formulation for the mucosal treatment of a disease, comprising the gold nanoparticles of claim 1, in combination with a drug effective for the treatment of said disease, wherein said mucosal treatment is on a mucous membrane.

23. The formulation of claim 22, capable of being administered topically, orally or in aerosol.

24. The formulation for the mucosal treatment of a disease of claim 22, wherein said mucosal treatment is adapted to be effective with a mucous membrane.

25. The formulation for the mucosal treatment of a disease of claim 24, wherein said mucous membrane is selected from the group consisting of: ocular or ophthalmic mucous membrane, bronchial mucosa, endometrium, esophageal mucosa, gastric mucosa, intestinal mucosa, nasal mucosa, oral mucosa, penile mucosa, vagina mucosa and anal mucosa.

26. The formulation for the mucosal treatment of a disease of claim 25, wherein said mucous membrane is ocular or ophthalmic mucous membrane.

27. The formulation for the mucosal treatment of a disease of claim 26, wherein said disease is selected from: glaucoma, macular degeneration, blepharitis, conjunctivitis, dry eye syndrome, eye allergies, and eye infections.

28. (canceled)

29. The formulation for the mucosal treatment of a disease of claim 27, wherein said drug is travoprost or timolol.

30. A method for the treatment of a disease or disorder, comprising mucosally administering a therapeutic dose of the formulation of claim 22.

31. A method for the treatment of glaucoma comprising the step of:
topically administering to a cornea, a dose of a formulated drug comprising about 0.004% of travoprost formulated with gold nanoparticles as defined in claim 1.

32. The method of claim 31, wherein said formulated drug is administered at an interval of less than once per day.

33. The method of claim 32, wherein said formulated drug is administered no more often than every two days.

34-41. (canceled)

42. A method for synthesizing gold nanoparticles, said method comprising the steps of:
a) providing chloroauric acid (HAuCl₄) dissolved in a mix acetonitrile/isopropyl alcohol to form a gold solution;
b) providing PEG-SH ligand dissolved in isopropyl alcohol;
c) adding said dissolved PEG-SH ligand to said gold solution to form a first mixture;
d) agitating said first mixture;
e) adding dropwise NaBH₄ dissolved in water to the first mixture under intense agitation to form a second mixture;
f) leaving said second mixture for sufficient time to stabilize;
g) evaporating said acetonitrile/isopropyl alcohol from said second mixture to obtain an aqueous solution of gold nanoparticles; and
h) submitting said aqueous gold nanoparticles solution to dialysis to remove excess PEG-SH ligand.

43-50. (canceled)

51. The method of claim 42, comprising the steps of:
a′) dissolving chloroauric acid (HAuCl₄) in an acetonitrile-containing solvent to form a partially-reduced gold solution;
b′) dissolving biocompatible ligand-SH in an organic solvent;
c′) adding said dissolved biocompatible ligand-SH to said partially-reduced gold solution to form a partially-reduced mixture;
d′) reducing the partially-reduced mixture to form a reduced mixture; and
e′) leaving said reduced mixture for sufficient time to proceed to nucleation and crystallization.
